(19) United States
(12) Patent Application Publication
(10) Pub. No.: US 2024/0412369 A1
(43) Pub. Date: Dec. 12, 2024

(54) METHOD FOR DETERMINING THE SUSCEPTIBILITY OF A MICROORGANISM TO AN ANTIMICROBIAL AGENT

(71) Applicant: BIOMERIEUX, Marcy L'Etoile (FR)

(72) Inventors: Bruno TETART, Craponne (FR); Nicolas FAURE, Grenoble (FR); Denis LEROUX, Trevoux (FR); Jean-Marc ROCHE, Feissons Sur Salins (FR); Jordane LALLEMAND, Uchaud (FR)

(73) Assignee: BIOMERIEUX, Marcy L'Etoile (FR)

(21) Appl. No.: 18/712,135

(22) PCT Filed: Nov. 24, 2022
(86) PCT No.: PCT/FR2022/052171

§ 371 Date: May 21, 2024

(30) Foreign Application Priority Data
Nov. 24, 2021 (FR) FR2112454

(51) Int. Cl. G06T 7/00; G06V 10/82; G06V 20/69

(52) U.S. Cl. CPC G06T 7/0014; G06V 10/82; G06V 20/698; G06T 2207/20081; G06T 2207/20084

(57) ABSTRACT

The present invention relates to a method for predicting the susceptibility of a microbial strain to an antimicrobial agent, the method being characterized in that it comprises implementation, by data-processing means (4) of a client (2), of steps of: (a) obtaining a hyperspectral image between 390 nm and 900 nm representing at least one colony of said strain in a sample devoid of antimicrobial agent (22); (b) determining a spectrum of the colony based on pixels of said hyperspectral image corresponding to said colony, “test spectrum” below; (c) comparing said test spectrum with microbial classes of a database of predetermined data, “reference microbial class” below, said classes corresponding to a taxonomic level lower than species and being learnt using at least one hyperspectral spectrum of a microbial strain, the database containing, for each reference microbial class, the susceptibility to the microbial agent of the reference microbial class; (d) determining the susceptibility of the microbial strain to the microbial agent to be the susceptibility associated with the reference microbial class closest to the test hyperspectral spectrum.

### CROSS-REFERENCE TO RELATED APPLICATIONS

This application is a national phase entry of PCT Patent Application Serial No. PCT/FR2022/052171, filed on Nov. 24, 2022, which claims priority to the French Patent Application Serial No. FR2112454, filed Nov. 24, 2021, both of which are incorporated by reference herein.

### TECHNICAL FIELD

The invention relates to the field of microbiological analysis, and in particular to the characterization of microorganisms, notably the prediction of the sensitive or resistant nature of yeasts, molds and bacteria to an antimicrobial agent. Advantageously, the invention applies to the analysis of a hyperspectral image of one or more colonies of bacteria, molds or yeasts that have grown in an observable culture medium.

### BACKGROUND

In the field of in vitro diagnosis of microorganisms, in particular pathogens, characterizing a microorganism preferably involves identifying its species and its sensitivity to an antimicrobial agent, (or “antibiogram”), in order to determine a treatment for the patient infected with this microorganism. To this end, a complex microbiological process is usually implemented in a laboratory, which process most often requires prior knowledge of other properties of the microorganism, notably its regnum (for example, yeast or bacterium), and, within the bacterial context, its Gram type or its fermentative or non-fermentative nature. Indeed, this information notably allows a culture medium or a type of antimicrobial agent to be selected that is adapted to the microorganism in order to ultimately determine its species or its antibiogram. For example, the selection of an API® microorganism identification gallery marketed by the Applicant is based on knowledge of the regnum of the microorganism (for example, yeast vs bacterium) or of the Gram type of the bacterial strain to be identified. Similarly, determining the antibiogram of a bacterial strain using the Vitek® 2 system marketed by the Applicant is based on the selection of a card as a function of the Gram type and of the fermentative or non-fermentative nature of said strain. It is also possible to cite identification by MALDI-TOF mass spectrometry using a different matrix depending on whether the microorganism to be identified is a yeast or a bacterium. Thus, knowing this information as soon as possible optimizes the microbiological process, notably by accelerating said process or by reducing the amount of consumables used.

Historically, each of these properties is determined using a technique that includes a significant number of manual steps (attachment, staining, mordanting, washing, over staining, etc.), and is therefore time consuming to implement. International application WO 2019/122732 describes a method for determining the Gram type and the fermentation nature of a bacterium strain that is automatic and that does not require marking or staining the bacterium or its culture medium in order to determine these features. To this end, an imaging system is used that is referred to as a multispectral or even hyperspectral imaging system. This is a system with high spectral resolution allowing the production of a digital image of the light reflected by, or transmitted through, the Petri dish with a significant number of channels. While a standard RGB image has three channels, an HSI (“Hyper Spectral Imaging”) image forms a data cube that can have several hundred spectral channels over a wavelength range of 390 to 900 nm (that is a spectral resolution of a few nanometers). A suitable classification algorithm applied to the HSI image then allows the type of Gram and the fermentative or non-fermentative nature of the represented strain to be determined. A culture medium or a type of antimicrobial agent suitable for the microorganism then can be selected in order to ultimately determine its sensitivity to the antibiotic as a function of its growth in a sample of the culture medium.

The document by Arrigoni, Turra and Signoroni, entitled, “Hyperspectral image analysis for rapid and accurate discrimination of bacterial infections: A benchmark study”, even proposes directly determining the species of the microorganism from the HSI image. As explained, this information is interesting, but is not sufficient in itself for determining whether the microorganism is resistant to an antimicrobial, and the antibiogram still needs to be produced. Indeed, for the same species, such as S. aureus, some strains are resistant while others are not resistant. For example, MRSA (Methicillin-resistant Staphylococcus aureus) and MSSA (Methicillin-sensitive Staphylococcus aureus) are referred to, i.e., strains of S. aureus that are respectively resistant or non-resistant to the methicillin antibiotic.

The document by Park et al., entitled, “Classification of Salmonella Serotypes with Hyperspectral Microscope Imagery”, proposes a solution for classifying microorganisms at a taxonomy lower than the species, yet to the detriment of complex handling and materials. Indeed, a colony needs to be isolated, then an HSI image of this colony needs to be specifically acquired using an “HMI” microscope. The algorithm then observes the cells on an individual scale and one by one, with this individual observation being used for the classification.

It thus remains desirable to be able to have a fast and efficient solution for determining the susceptibility, i.e., the resistance or the sensitivity, of a microorganism to an antimicrobial agent. Such a solution is integrated, for example, in a clinical process involving taking the sample from a patient likely to be infected with a pathogenic microorganism, preparing the sample for analysis using the solution of the invention, applying the solution of the invention, selecting an antimicrobial as a function of the susceptibility result provided by the solution, and then applying the selected antimicrobial to the patient. Advantageously, the invention is applicable to the analysis of a hyperspectral image of one or more colonies of bacteria, of molds or of yeasts that have grown in a culture medium and that can be observed without using markers or staining, without observing cells on an individual scale or without using a high magnification optical system such as a microscope, and without having to destroy bacteria or colonies. Advantageously, the invention is applicable as soon as a colony occupies some pixels in the acquired hyperspectral image, notably from 10 pixels.

### SUMMARY

The aim of the present invention is to predict the susceptibility of a microorganism to an antimicrobial agent using hyperspectral imaging of a microbial colony that has grown on a culture medium without the presence of said antimicrobial agent. To this end, the aim of the invention is a method for predicting the susceptibility of a microbial strain to an antimicrobial agent, the method being characterized in that it comprises implementing, by data processing means of a client, the following steps:
(a) obtaining a hyperspectral image between 390 nm and 900 nm representing at least one colony of said strain in a sample devoid of an antimicrobial agent;
(b) determining a spectrum, called “test spectrum” hereafter, of the colony from the pixels of said hyperspectral image corresponding to said colony;
(c) comparing said test spectrum with microbial classes, called “reference microbial class” hereafter, of a predetermined database, with said classes corresponding to a taxonomic level lower than the species and being learnt on at least one hyperspectral spectrum of a microbial strain, the database comprising, for each reference microbial class, the susceptibility to the antimicrobial agent of the reference microbial class;
(d) determining the susceptibility of the microbial strain to the microbial agent as being that associated with the reference microbial class closest to the hyperspectral test spectrum.

In other words, the inventors have discovered that hyperspectral imaging between 390 nm and 900 nm contains enough information to predict that two microbial strains are clonal or are derived from the same line and thus share the same susceptibility to the antimicrobial agent. By knowing the susceptibility of a class, by predicting that a new microorganism belongs to said class, the new microorganism is able to predict the susceptibility of said class.

The term “microbial class” is understood herein to mean any digital object characterizing the microbial identity on a taxonomic level lower than the species, and notably on a strain level, with which object the hyperspectral spectrum of a colony can be compared using a suitable metric in order to determine whether or not said colony belongs to said class. The microbial classes can be classes learnt by monitored or non-monitored machine learning algorithms, or by reference hyperspectral spectra, for example.

According to a preferred embodiment, the steps of comparing and of determining are carried out by means of a predictor based on a monitored classification having the identity of the microbial strains of the database as reference microbial classes, with the phase of training the classification comprising:
(c1) acquiring hyperspectral spectra of various colonies for each microbial strain of the database;
(c2) training the classification on the hyperspectral spectra of various colonies.

In other words, rather than determining a spectrum representing a microbial strain that would be compared with the spectrum of a colony undergoing testing, this embodiment learns the classes from hyperspectral spectra derived from various colonies of the microbial strain, which allows any variation in the acquisition of spectra to be taken into account, such as the measurement error, the variability of the lighting or even the variability of the spectrum with a biological nature (variable thickness of the colonies modifying the spectra, variable colors, etc.).

More specifically, the predictor is a convolutional artificial neural network. Preferably, the database is frequently updated in order to take into account new strains not yet listed, intra-strain variability of the hyperspectral spectra or in order to incorporate data resulting from the preparation of samples and different lighting. The use of such a predictor provides processing flexibility since the pre-processing it incorporates (for example, extracting features by reducing the size of the variables by the one or more convolutional layers) is not set a priori.

According to embodiments of the invention:
step (b) comprises segmenting said hyperspectral image so as to detect said colony in the sample;
step (a) comprises acquiring said hyperspectral image by an observation device connected to said client device;
the method comprises a step (a0) of learning, by data processing means of a server, parameters of said automatic classification model from a training database of hyperspectral images or of already classified spectra of colonies;
the microbial strain is a Staphylococcus aureus strain and the antimicrobial agent is methicillin.

A further aim of the invention is a system for determining the susceptibility of a microorganism to an antimicrobial agent, comprising at least one client device comprising data processing means, characterized in that said data processing means are configured to implement:
obtaining a hyperspectral image representing at least one colony of said microorganism in a sample;
determining a spectrum of the colony from the pixels of said hyperspectral image corresponding to said colony;
comparing said test spectrum with microbial classes, called “reference microbial class” hereafter, of a predetermined database, with said classes corresponding to a taxonomic level lower than the species and being learnt on at least one hyperspectral spectrum of a microbial strain, the database comprising, for each reference microbial class, the susceptibility to the antimicrobial agent of the reference microbial class;
determining the susceptibility of the microbial strain to the microbial agent as being that associated with the reference microbial class closest to the hyperspectral test spectrum.

According to one embodiment, the system further comprises an observation device for acquiring said hyperspectral image. A further aim of the invention is a computer program product comprising code instructions for executing a method as described above for determining the susceptibility of a microorganism to an antimicrobial agent, when said program is executed on a computer. A further aim of the invention is a computer device-readable storage means storing a computer program product comprising code instructions for executing a method as described above for determining the susceptibility of a microorganism to an antimicrobial agent.

### DESCRIPTION OF THE DRAWINGS

Further features and advantages of the present invention will become apparent upon reading the following description of a preferred embodiment. This description will be provided with reference to the appended drawings, in which:

FIG. 1 is a diagram of an architecture for implementing the method according to the invention;

FIG. 2a shows a first embodiment of a device for observing microorganisms in a sample used in an embodiment of the method according to the invention;

FIG. 2b shows a second embodiment of a device for observing microorganisms in a sample used in a preferred embodiment of the method according to the invention;

FIG. 3a shows an example of a colony spectrum of a class with resistance to an antimicrobial agent;

FIG. 3b shows an example of a colony spectrum of a class with sensitivity to an antimicrobial agent;

FIG. 4 shows the steps of a preferred embodiment of the method according to the invention;

FIG. 5 shows an example of a convolutional neural network architecture used in a preferred embodiment of the method according to the invention; and

FIG. 6 shows a confusion matrix of a predictor based on a convolutional neural network according to the invention.

### DETAILED DESCRIPTION

### Architecture:

The invention relates to a method for determining the susceptibility of a microorganism of a given species to an antimicrobial agent. Said microorganism is typically a bacterium, a mold or a yeast (the example of S. aureus will be used throughout the remainder of the description, but this could be E. coli, C. difficile, etc.), and said microbial agent is an antibiotic (in particular methicillin was then the antibiotic of choice for S. aureus, but also vancomycin, for example) or an antifungal agent relating to yeasts and molds.

As will be seen, this method can include a machine learning component, and notably a classification model selected from among a support vector machine (SVM) or a convolutional neural network (CNN). More specifically, the method is a method for classifying an image, called hyperspectral image, of the microorganism, such that the input or learning data are of the image type, and represent at least one colony of said microorganism in a sample 22 (in other words, it involves images of the sample in which at least one colony, generally a plurality of colonies, is visible, i.e., detectable to the naked eye by a laboratory technician or detectable in the image by means of a segmentation algorithm that is per se known. By way of an example, a colony is detectable as soon as it reaches a size of more than 10 pixels in the image). The sample 22 is adapted to the culture of said microorganism, typically an agar poured in a Petri dish, even though it can be any culture medium or reactive medium. The notion of a hyperspectral image, denoted HSI image, will be referred to hereafter.

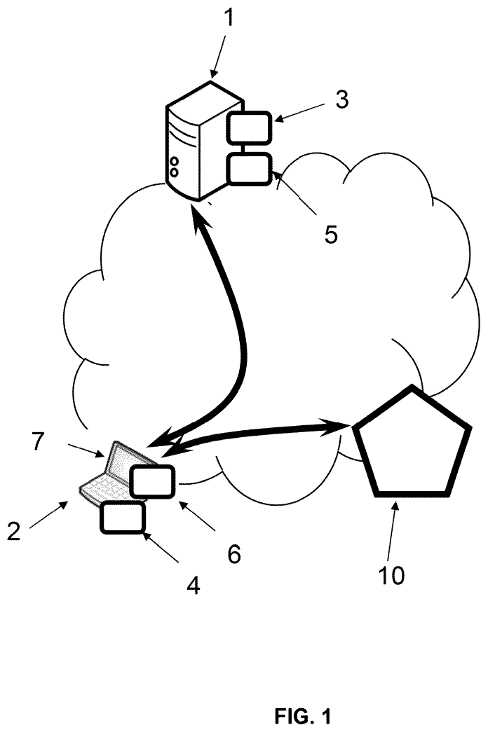

The present methods are implemented within an architecture as shown in FIG. 1, by virtue of a server 1 and a client device 2. The server 1 is the learning device (implementing the learning method) and the client device 2 is an operating device (implementing the method for determining the susceptibility of a microorganism to an antimicrobial agent), for example, a terminal of a doctor, a hospital or a microbiology laboratory.

It is quite possible for the two devices 1, 2 to be merged, but preferably the server 1 is a remote device, and the client device 2 is a consumer device, notably a desktop computer, a laptop, etc. The client device 2 is advantageously connected to an observation device 10, so as to be able to directly acquire said input image, typically for processing it directly; alternatively, the input image will be loaded on the client device 2.

In all cases, each device 1, 2 is typically a remote computing device linked to a local network or to a wide area network, such as the Internet, for exchanging data. Each device comprises data processing means 3, 4 of the processor type, and data storage means 5, 6, such as a computer memory, notably a permanent memory, for example, a flash memory or a hard disk, storing all the computer instructions for implementing the method according to the invention. The client device 2 typically comprises a user interface 7, such as a screen, for interaction.

The server 1 advantageously stores a database for the considered species, comprising a list of microbial strains belonging to the species, and, for each of said strains, comprising:
hyperspectral spectra for learning colonies of the strain, i.e., a set of already classified objects;
data relating to the sensitive or resistant nature of the strain to the microbial agent;
optionally data relating to the test conditions.

### Acquisition:

Even if, as explained, the present method can directly take any hyperspectral image as input representing at least one colony of said microorganism in the sample 22, in particular a Petri dish in which an agar is poured forming a nutrient medium allowing the growth of microbial colonies after spreading a liquid sample containing one or more microbial strains, obtained in any manner, the present method preferably begins with a step (a) of obtaining the input image from data supplied by an observation device 10. In a known manner, a person skilled in the art can use hyperspectral imaging techniques, in particular as described in international application WO 2019/122732.

A hyperspectral image is understood to mean an image comprising a large number of spectral channels, in particular at least seven, advantageously at least twenty, and potentially more than two hundred (the example of 223 channels will be used), compared with a conventional three-channel RGB image. In general, the device 10 is “simple” compared to that notably described by Park et al., in the document entitled, “Classification of Salmonella Serotypes with Hyperspectral Microscope Imagery”, in that it simply needs to be able to acquire an HSI image of the sample 22, and therefore does not require a microscope, the high magnification of which makes focusing difficult.

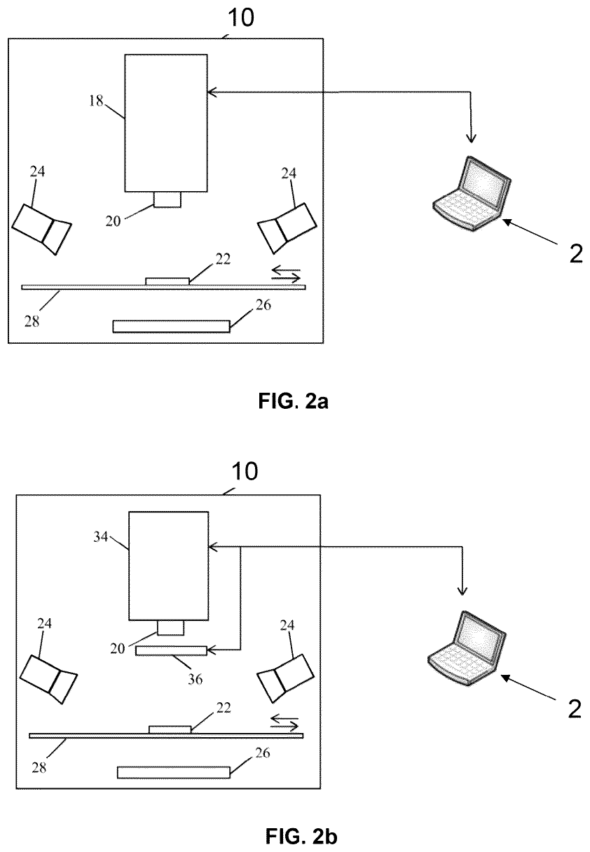

Two possible embodiments of the device 10, corresponding to FIGS. 2a and 2b, will now be described.

With reference to FIG. 2a, the device 10 is, for example, a reference hyperspectral imaging system, namely “Pika II” marketed by Resonon, Montana, USA. It advantageously comprises:
a “hyperspectral’ camera 18, made up of a digital sensor comprising an array of elementary sensors, for example, a CCD or CMOS type digital sensor, sensitive in a wavelength range, for example, [λmin; λmax]=[390 nm; 900 nm]; and of a light dispersive element or a spectrograph for selecting a wavelength to be acquired by the sensor;
an objective lens 20 for focusing, on the digital sensor of the camera 18, the optical image of the sample 22 for which a hyperspectral image is to be acquired;
front lighting 24, for example, made up of one or more allogeneic lamps, for example, 2 or 4 lamps, able to emit light in the range [Amin; Amax] and to provide uniform front lighting of the sample 22. For example, the lighting is of the white light lamp type;
rear lighting 26, for example, made up of a matrix of white light LEDs, in order to provide uniform rear lighting of the sample 22 in the range;
a carriage 28 supporting the sample 22 and allowing the sample to pass in front of the objective lens 20 in order to obtain a complete image by scanning.

The device 10 is configured, for example, to acquire the image of a region measuring 90 millimeters by 90 millimeters with a sampling rate of 160 micrometers (spatial resolution estimated at 300 micrometers) and with a spectral resolution of a few nanometers over the range [λmin; λmax]. 200 channels can be exceeded over a range of approximately 500 nm. In particular, the field of view and the depth of field of the objective lens 20 are selected so as to obtain images that can include complete colonies with a radius of up to 1 cm, preferably of up to 0.9 cm, and even more preferably of 0.5 cm.

The device 10 thus produces a digital HSI image of the light reflected by the sample 22, incorrectly called “hypercube” since it is actually three-dimensional: two spatial dimensions and one spectral dimension, with each pixel (or rather voxel due to the three-dimensional nature of the HSI image) representing the radiance measured at a point of the sample 22 for a spectral channel. The radiance of a pixel, commonly called “luminous intensity”, in this case corresponds to the amount of incident light on the surface of the corresponding elementary sensitive site of the sensor of the camera 18 throughout the exposure duration, as is known per se in the field of digital photography, for example. The device 10 can comprise on-board data processing means configured to process the HSI images produced by the camera 18 and/or to delegate everything to the client device 2.

These processing means in all cases are provided with the set of memories (RAM, ROM, cache, mass memory, etc.) for storing the images produced by the device 10, computer instructions for implementing the method according to the invention, parameters useful for this implementation and for storing the results of the intermediate and final computations. The client device 2 optionally comprises, as explained, a display screen 7 for displaying the final result of the method. Although a single processing unit is described, the invention obviously applies to processing carried out by several processing units (for example, an on-board unit in the camera 18 for pre-processing HSI images and the unit 4 of the client device 2 for implementing the remainder of the processing). Moreover, the interface 7 of the client device 2 can allow data to be entered that relates to the sample 22, notably the type of culture medium used when the prediction depends on the medium, for example, by means of a keyboard/mouse and a drop-down menu available to the operator, a barcode/QR code reader reading a barcode/QR code present on the Petri dish and comprising information relating to the sample 22, etc.

With reference to FIG. 2b, according to the second embodiment, the device 10 can alternatively comprise a camera 34, advantageously a high spatial resolution CMOS or CCD camera, coupled to a set of spectral filters 36, for example, disposed in front of the objective lens 20 between the objective lens 20 and the sensor of the camera 32. The set of filters 36 is made up of a number NF of distinct bandpass filters, each configured to only transmit light in part of the range [λmin; λmax], with a full width half maximum (FWHM) spectral width that is less than or equal to 50 nm, and preferably less than or equal to 20 nm. The set 36 is, for example, a filter wheel that can typically accommodate up to twenty-four different filters, which wheel is controlled by the data processing unit, which actuates it in order for said filters to pass in front of the camera, and to control image capturing for each of said filters.

### Method:

The “classification” of an input HSI image involves determining at least one class from among a set of possible classes describing the images. The present method proposes using an automatic classification model for determining whether the microorganism to be tested belongs to one of the strains already listed in the database and not for directly determining the susceptibility of the microorganism to the antimicrobial agent or even whether the microorganism belongs to particular stereotypes, for example.

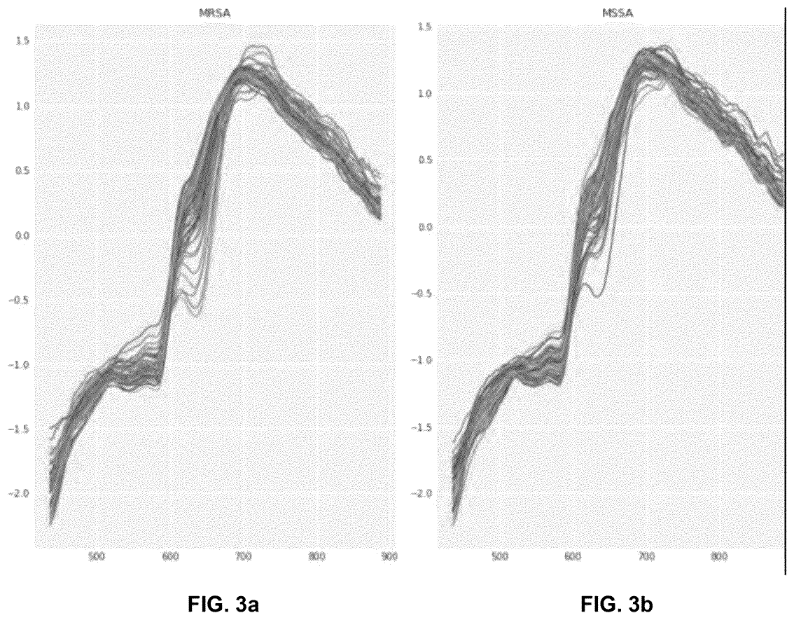

In particular, with reference to FIGS. 3a and 3b, which each represent a plurality of examples of spectra respectively for colonies of S. aureus resistant to methicillin (MRSA) and sensitive to methicillin (MSSA), it should be noted that the two spectra do not have exactly the same appearance, hence the possibility of a direct discriminating classification of the sensitive or resistant nature of a new colony of S. aureus. However, the inventors have found that the performance capabilities of such a susceptibility predictor are not sufficient in the field of clinical or industrial microbiology. For example, the performance capabilities of a Gaussian kernel SVM predictor plateau at 70% in BCR.

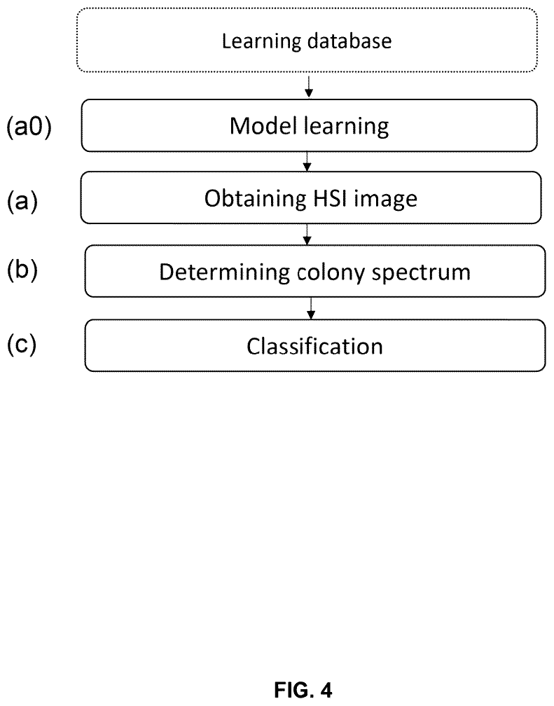

With reference to FIG. 4, the method comprises, after step (a) of obtaining the hyperspectral image, a step (b) of determining a spectrum of the colony from the pixels of said hyperspectral image corresponding to said colony. The term “spectrum of the colony” is understood to mean a curve representing the light intensity measured to the scale of the colony as a function of the frequency. Mathematically, this is a size vector relating to the number of channels of the HSI image (i.e., 223 in the example provided herein). Preferably, this spectrum is determined as the mean spectrum on the pixels of said hyperspectral image corresponding to said colony. Indeed, by way of a reminder, the HSI image includes a plurality of corresponding intensity values for each spatial pixel.

In this respect, step (b) advantageously comprises segmenting said hyperspectral image so as to detect said colony in the sample 22, then determining the spectrum, as is typically explained by averaging the intensity on a channel by channel basis over the segmented pixels. For example, step (b) comprises automatically detecting colonies (for example, by applying a filter selecting the round objects in the image, for example, a Hough transformation), and/or a manual step of selecting colonies by a laboratory technician, for example. In other words, there is a size vector of n=223 per pixel of the colony, and the mean of these vectors is made into a vector representing the colony. In practice, a colony generally extends over a zone of the HSI image with a maximum size of 11×11, so that the mean only needs to be provided for around a hundred vectors.

In the case whereby the hyperspectral image represents a plurality of colonies of said microorganism, the method according to the invention can be applied to each colony or to a set of colonies selected according to criteria relating to the size or the position in the culture medium, for example. In general, the segmentation allows all the colonies of interest to be detected, by removing artefacts such as filaments or dust. The segmentation can be implemented in any known manner.

Step (b) advantageously comprises processing the spectrum, in particular smoothing and/or normalizing the spectrum:
smoothing involves removing the peaks that are probably artefacts, for example, by determining the moving mean;
normalizing aims to make the spectra comparable, notably using a standard normal variate (SNV) technique involving subtracting the mean from the spectrum and dividing it by its standard deviation.

It should be noted that if the learning database directly stores reference spectra, they preferably must have undergone the same smoothing and/or normalizing, if necessary.

### Classification:

In a step (c), said spectrum of the colony (if necessary smoothed and/or normalized) is, as explained, directly classified by means of an automatic classification model from among a microbial class made up of the identity of the strains listed in the database. If a plurality of spectra has been determined, each spectrum can be classified, and the results can be aggregated. The automatic classification model can be, as explained, a support vector machine (SVM) or a convolutional neural network (CNN). In the case of an SVM, an RBE (Radial Basis Function) kernel SVM is selected, for example.

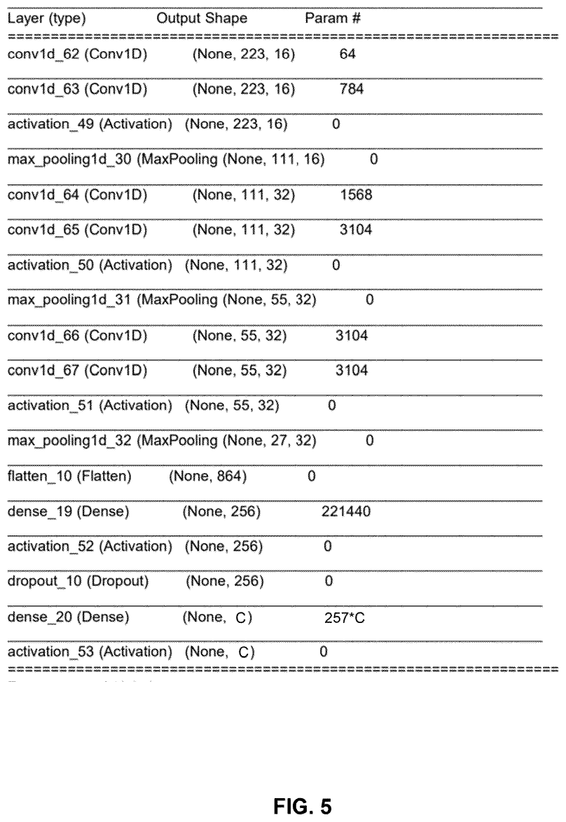

In the case of a CNN, an architecture of the type shown in FIG. 5 is selected, for example, which is particularly suitable for implementing the present method. Conventionally, this architecture advantageously comprises a series of “convolutional blocks” made up of one or more 1D convolutional layers (since the input is not an image but the spectrum, i.e., a one-dimensional object), an activation layer (for example, the ReLU function) for increasing the depth of the feature maps, and a 1D pooling layer (in this case max pooling) allowing the size of the feature map to be reduced (generally by a factor of 2). It is noteworthy that two convolutional blocks can suffice, so that, highly preferably, the present CNN includes only two convolutional blocks.

Thus, in the example of FIG. 5, the CNN starts, as explained, with 12 layers distributed in 3 blocks. The first block takes the spectrum as input (thus forming a 223-size object), and includes a dual convolution+activation sequence increasing the depth to 16 and then a max pooling layer (it is also possible to use overall average pooling), with a 111×16-size feature map as output (the size is divided by two according to the spectral dimension). The second block has an architecture identical to the first block and generates, as output from a new dual convolution+activation, a 111×32-size feature map (depth doubled) and, as output from the max pooling layer, a 55×32-size feature map (with a new reduction in the spectral size by a factor of two). The third block has an architecture identical to the first two blocks and generates, as output from a new dual convolution+activation, a 55×32-size feature map (depth unchanged) and, as output from the max pooling layer, a 27×32-size feature map (with a new reduction in the spectral size by a factor of two).

At the output of the last convolutional block (in this case the third block), the CNN advantageously comprises a “flattening” layer that transforms the final feature map (containing the most “in-depth” information) output from this block into a vector (1-dimensional object). Thus, for example, the 27×32-size feature map switches to a vector that is 27*32=864. It will be understood that there is no limit to the sizes of maps/filters on any level, and that the aforementioned sizes are only examples.

Finally, conventionally, this results in one or more fully connected layers (FC, or “dense” layers, as indicated in FIG. 5) and optionally a final activation layer, for example, a softmax layer. In the example shown, a first dense layer transforms the 864-size vector into a smaller 256-size vector (which requires (864+1)*256=22, 1440 parameters, that is 90% of all the parameters of the CNN), and a second FC layer transforms the 864-size vector into a final vector of size C, with C being the desired total number of classes, that is 2, 5 or 11 in the aforementioned examples (which requires (256+1)*C parameters).

Preferably, the CNN is made up of (i.e., includes exactly) a sequence of convolutional blocks, then a flattening layer, and finally one or more fully connected layers. Therefore, it can be seen that the total number of parameters is of the order of 200,000, which is remarkably low for a CNN (commonly there are several tens of millions of parameters). The present CNN therefore can be used by many client devices 2, including client devices with moderate computing resources. Again, it should be noted that the term “direct classification” or “end-to-end” is understood to mean without pre-classification or separate extraction of at least one feature map of said colony: it is understood that the CNN naturally has internal states in the form of feature maps, but these maps are never returned to the outside of the CNN, with the CNN only having the result of the classification as output.

### Learning:

Preferably, the method can comprise a step (a0) of learning, by the data processing means 3 of the server 1, the parameters of the automatic classification model from a learning database. Indeed, this step is typically implemented well upstream, in particular by the remote server 1. As explained, the learning database can include a certain amount of learning data, in particular hyperspectral images of colonies or even directly from the spectra, in all cases associated with their class (i.e., the identity of the microbial strains).

The learning for the model can be carried out in any way known to a person skilled in the art that is adapted to the selected model. In all the embodiments, the parameters of the learnt model can be stored, if necessary, on data storage means 21 of the client device 2 for use in classification. It should be noted that the same model can be included on many client devices 2, yet only one learning step is necessary.

Preferably, the learning database for the considered microbial species is formed as follows. The following is carried out for each strain of said species:
producing several samples, preferably at least 3 samples, for example, several Petri dishes in which a nutrient agar has been poured that contains neither the antimicrobial agent nor a marking or staining agent, on which agar colonies of said strain have grown;
acquiring and storing hyperspectral spectra of at least one colony of each sample using a device as described in FIG. 2, preferably of several colonies per Petri dish and arranged at different distances from the center of the Petri dishes, and processing said spectra as described above. The spectra of the first two samples are used for learning the microbial classes of strains, with the spectra of the other samples (for example, the third sample when producing replicates) being used to test the performance capabilities of the learning. Optionally, the acquisition is also carried out on several samples using different capturing devices for capturing the variability of the spectra caused by differences in the features of the devices (for example, the variability of the light sources between devices, etc.);
phenotypic measuring of the susceptibility of the strain to the antimicrobial agent, for example, by means of a Vitek® 2 marketed by the Applicant or the use of an e-test or of a diffusion disc as is well known per se in the prior art, and the storage thereof;
genomic characterizing of the strain, for example, using complete sequencing of its genome and establishing a wgMLST profile as described in the document entitled, “MLST revisited: the gene-by-gene approach to bacterial genomics”, by Martin C. J. Maiden, Nature Reviews Microbiology, 2013, and storing this characterization.

### Updating the Database and the Predictor:

When a strain is not listed in the database, as determined, for example, by the predictor according to the invention, which returns an uncertain classification in the pre-learnt microbial strain classes, characterizing said strain as described above is advantageously carried out. The genomic profile of the strain is advantageously compared with the stored genomic profiles in order to determine whether it is actually a strain different from those stored in the database. In this case, the data gathered for the strain are stored in the database and new learning is carried out as described above in order to incorporate a new microbial class corresponding to the unlisted strain.

### Computer Program Product:

According to a second and a third aspect, the invention relates to a computer program product comprising code instructions for executing (in particular on the data processing means 3, 5 of the server 1 and/or of the client device 2) a method for determining the susceptibility of a microorganism to an antimicrobial agent, as well as computer device-readable storage means (a memory 4, 6 of the server 1 and/or of the client device 2) on which this computer program product is found.

### Preferred Applications:

The invention is advantageously incorporated in:
a method for epidemiologically monitoring strains of interest in a clinical or industrial environment (for example, a hospital department, a hospital as a whole, a group of hospitals, an agri-foodstuff plant, a potable water distribution facility, etc.) defining a given geographical zone, the database being made up of the strains sampled from said zone. The invention thus provides two important items of information for fighting nosocomial diseases or microbial contaminations not in accordance with food, environmental or manufacturing standards: the identification or non-identification of a newly sampled strain with a strain already seen in the geographical sampling zone and its susceptibility to the antimicrobial agent. These data associated with the sampling zones (for example, hospital room, production zone) allow, for example, investigations to be conducted concerning how these germs are disseminated within the hospital or within the production plant. It should be noted that within the context of hospital epidemiological monitoring, all the cultures can be used to feed the knowledge database, those implemented within the context of a microbiological diagnosis process, whether or not it integrates a susceptibility test, as well as those implemented in a hospital admission screening process on prevention range media. This invention thus provides the possibility of carrying out epidemiological monitoring of the various resistance clones present in the hospital;
a method for antibiotic therapy of a patient suspected of being infected with a pathogen. As is known per se, when a patient is suspected of being the victim of an infection, a combination of a broad spectrum of antibiotics is generally administered to them before knowing the identity and the antibiogram of the strain they are infected with, with the antibiotic therapy then being optionally modified once the antibiogram of the strain has been carried out. A microbial strain is usually characterized by proceeding through several successive steps of growing colonies on a Petri dish. By virtue of the invention, from the first sign of growth, a prediction of the identity of the strain and its susceptibility to one or more antimicrobials is available so that the clinician can adapt their therapy without waiting for the result of an antibiogram.

### EXAMPLE

The invention has been applied to predicting the susceptibility of 50 strains of Staphylococcus aureus to methicillin so as to define a predictor based on CNN identifying the MRSA and MSSA strains. The following table specifies, for each of the strains listed in the learning database, the number of colonies for which hyperspectral spectra were acquired and the susceptibility to methicillin.

| API number | Susceptibility | Total number |
|---|---|---|
| of the strain | to methicillin | of colonies |
| 1412368 | MSSA | 145 |
| 1412369 | MSSA | 108 |
| 412370 | MSSA | 134 |
| 1502039 | MSSA | 188 |
| 1412148 | MRSA | 179 |
| 1412174 | MRSA | 129 |
| 1412289 | MRSA | 86 |
| 1412330 | MRSA | 111 |
| 1412343 | MRSA | 118 |
| 1412350 | MRSA | 89 |
| 1502059 | MSSA | 139 |
| 1502063 | MSSA | 159 |
| 1502098 | MRSA | 187 |
| 1502125 | MSSA | 170 |
| 1412147 | MSSA | 128 |
| 1412162 | MSSA | 153 |
| 1412277 | MRSA | 116 |
| 1412300 | MRSA | 140 |
| 1412315 | MRSA | 166 |
| 1412316 | MRSA | 127 |
| 1502094 | MSSA | 209 |
| 1502121 | MSSA | 205 |
| 1503077 | MRSA | 142 |
| 1503078 | MRSA | 164 |
| 1412156 | MSSA | 213 |
| 1412157 | MSSA | 186 |
| 1412202 | MSSA | 78 |
| 1502123 | MSSA | 142 |
| 1412145 | MSSA | 161 |
| 1412153 | MSSA | 140 |
| 1412170 | MRSA | 200 |
| 1412171 | MSSA | 127 |
| 1412260 | MRSA | 108 |
| 1412262 | MRSA | 81 |
| 1412278 | MRSA | 122 |
| 1412303 | MRSA | 170 |
| 1412340 | MRSA | 123 |
| 1502073 | MSSA | 172 |
| 1412175 | MSSA | 128 |
| 1412177 | MSSA | 170 |
| 1502095 | MSSA | 206 |
| 1502124 | MSSA | 160 |
| 1412152 | MSSA | 125 |
| 1412197 | MSSA | 164 |
| 1412198 | MSSA | 148 |
| 1502041 | MSSA | 175 |
| 1412232 | MRSA | 154 |
| 1412233 | MRSA | 100 |
| 1412327 | MRSA | 91 |
| 1412353 | MRSA | 85 |

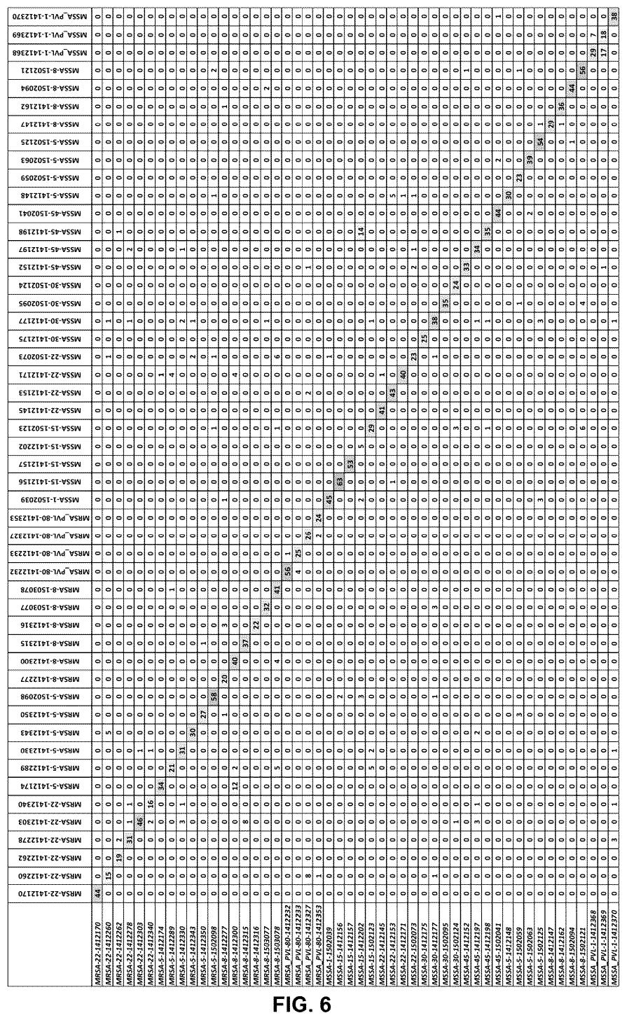

FIG. 6 illustrates the confusion matrix of a predictor of microbial strains of strains according to the neural network of FIG. 5. The global accuracy of the latter (global accuracy) is 88% and the mean accuracy per class (“balanced accuracy”) is 87%.

Method for determining the susceptibility of a microorganism to an antimicrobial agent

### GENERAL TECHNICAL FIELD

The invention relates to the field of microbiological analysis, and in particular to the characterization of microorganisms, notably the prediction of the sensitive or resistant nature of yeasts, molds and bacteria to an antimicrobial agent.

Advantageously, the invention applies to the analysis of a hyperspectral image of one or more colonies of bacteria, molds or yeasts that have grown in an observable culture medium.

### PRIOR ART

In the field of in vitro diagnosis of microorganisms, in particular pathogens, characterizing a microorganism preferably involves identifying its species and its sensitivity to an antimicrobial agent, (or “antibiogram”), in order to determine a treatment for the patient infected with this microorganism. To this end, a complex microbiological process is usually implemented in a laboratory, which process most often requires prior knowledge of other properties of the microorganism, notably its regnum (for example, yeast or bacterium), and, within the bacterial context, its Gram type or its fermentative or non-fermentative nature. Indeed, this information notably allows a culture medium or a type of antimicrobial agent to be selected that is adapted to the microorganism in order to ultimately determine its species or its antibiogram. For example, the selection of an API® microorganism identification gallery marketed by the Applicant is based on knowledge of the regnum of the microorganism (for example, yeast vs bacterium) or of the Gram type of the bacterial strain to be identified. Similarly, determining the antibiogram of a bacterial strain using the Vitek® 2 system marketed by the Applicant is based on the selection of a card as a function of the Gram type and of the fermentative or non-fermentative nature of said strain. It is also possible to cite identification by MALDI-TOF mass spectrometry using a different matrix depending on whether the microorganism to be identified is a yeast or a bacterium. Thus, knowing this information as soon as possible optimizes the microbiological process, notably by accelerating said process or by reducing the amount of consumables used.

Historically, each of these properties is determined using a technique that includes a significant number of manual steps (attachment, staining, mordanting, washing, over staining, etc.), and is therefore time consuming to implement.

International application WO 2019/122732 describes a method for determining the Gram type and the fermentation nature of a bacterium strain that is automatic and that does not require marking or staining the bacterium or its culture medium in order to determine these features. To this end, an imaging system is used that is referred to as a multispectral or even hyperspectral imaging system. This is a system with high spectral resolution allowing the production of a digital image of the light reflected by, or transmitted through, the Petri dish with a significant number of channels. While a standard RGB image has three channels, an HSI (“Hyper Spectral Imaging”) image forms a data cube that can have several hundred spectral channels over a wavelength range of 390 to 900 nm (that is a spectral resolution of a few nanometers). A suitable classification algorithm applied to the HSI image then allows the type of Gram and the fermentative or non-fermentative nature of the represented strain to be determined. A culture medium or a type of antimicrobial agent suitable for the microorganism then can be selected in order to ultimately determine its sensitivity to the antibiotic as a function of its growth in a sample of the culture medium.

The document by Arrigoni, Turra and Signoroni, entitled, “Hyperspectral image analysis for rapid and accurate discrimination of bacterial infections: A benchmark study”, even proposes directly determining the species of the microorganism from the HSI image. As explained, this information is interesting, but is not sufficient in itself for determining whether the microorganism is resistant to an antimicrobial, and the antibiogram still needs to be produced. Indeed, for the same species, such as S. aureus, some strains are resistant while others are not resistant. For example, MRSA (Methicillin-resistant Staphylococcus aureus) and MSSA (Methicillin-sensitive Staphylococcus aureus) are referred to, i.e., strains of S. aureus that are respectively resistant or non-resistant to the methicillin antibiotic.

The document by Park et al., entitled, “Classification of Salmonella Serotypes with Hyperspectral Microscope Imagery”, proposes a solution for classifying microorganisms at a taxonomy lower than the species, yet to the detriment of complex handling and materials. Indeed, a colony needs to be isolated, then an HSI image of this colony needs to be specifically acquired using an “HMI” microscope. The algorithm then observes the cells on an individual scale and one by one, with this individual observation being used for the classification.

It thus remains desirable to be able to have a fast and efficient solution for determining the susceptibility, i.e., the resistance or the sensitivity, of a microorganism to an antimicrobial agent. Such a solution is integrated, for example, in a clinical process involving taking the sample from a patient likely to be infected with a pathogenic microorganism, preparing the sample for analysis using the solution of the invention, applying the solution of the invention, selecting an antimicrobial as a function of the susceptibility result provided by the solution, and then applying the selected antimicrobial to the patient. Advantageously, the invention is applicable to the analysis of a hyperspectral image of one or more colonies of bacteria, of molds or of yeasts that have grown in a culture medium and that can be observed without using markers or staining, without observing cells on an individual scale or without using a high magnification optical system such as a microscope, and without having to destroy bacteria or colonies.

Advantageously, the invention is applicable as soon as a colony occupies some pixels in the acquired hyperspectral image, notably from 10 pixels.

### DESCRIPTION OF THE INVENTION

The aim of the present invention is to predict the susceptibility of a microorganism to an antimicrobial agent using hyperspectral imaging of a microbial colony that has grown on a culture medium without the presence of said antimicrobial agent.

To this end, the aim of the invention is a method for predicting the susceptibility of a microbial strain to an antimicrobial agent, the method being characterized in that it comprises implementing, by data processing means of a client, the following steps:
(a) obtaining a hyperspectral image between 390 nm and 900 nm representing at least one colony of said strain in a sample devoid of an antimicrobial agent;
(b) determining a spectrum, called “test spectrum” hereafter, of the colony from the pixels of said hyperspectral image corresponding to said colony;
(c) comparing said test spectrum with microbial classes, called “reference microbial class” hereafter, of a predetermined database, with said classes corresponding to a taxonomic level lower than the species and being learnt on at least one hyperspectral spectrum of a microbial strain, the database comprising, for each reference microbial class, the susceptibility to the antimicrobial agent of the reference microbial class;
(d) determining the susceptibility of the microbial strain to the microbial agent as being that associated with the reference microbial class closest to the hyperspectral test spectrum.

In other words, the inventors have discovered that hyperspectral imaging between 390 nm and 900 nm contains enough information to predict that two microbial strains are clonal or are derived from the same line and thus share the same susceptibility to the antimicrobial agent. By knowing the susceptibility of a class, by predicting that a new microorganism belongs to said class, the new microorganism is able to predict the susceptibility of said class.

The term “microbial class” is understood herein to mean any digital object characterizing the microbial identity on a taxonomic level lower than the species, and notably on a strain level, with which object the hyperspectral spectrum of a colony can be compared using a suitable metric in order to determine whether or not said colony belongs to said class. The microbial classes can be classes learnt by monitored or non-monitored machine learning algorithms, or by reference hyperspectral spectra, for example.

According to a preferred embodiment, the steps of comparing and of determining are carried out by means of a predictor based on a monitored classification having the identity of the microbial strains of the database as reference microbial classes, with the phase of training the classification comprising:
(c1) acquiring hyperspectral spectra of various colonies for each microbial strain of the database;
(c2) training the classification on the hyperspectral spectra of various colonies.

In other words, rather than determining a spectrum representing a microbial strain that would be compared with the spectrum of a colony undergoing testing, this embodiment learns the classes from hyperspectral spectra derived from various colonies of the microbial strain, which allows any variation in the acquisition of spectra to be taken into account, such as the measurement error, the variability of the lighting or even the variability of the spectrum with a biological nature (variable thickness of the colonies modifying the spectra, variable colors, etc.).

More specifically, the predictor is a convolutional artificial neural network. Preferably, the database is frequently updated in order to take into account new strains not yet listed, intra-strain variability of the hyperspectral spectra or in order to incorporate data resulting from the preparation of samples and different lighting. The use of such a predictor provides processing flexibility since the pre-processing it incorporates (for example, extracting features by reducing the size of the variables by the one or more convolutional layers) is not set a priori.

According to embodiments of the invention:
step (b) comprises segmenting said byperspectral image so as to detect said colony in the sample;
step (a) comprises acquiring said hyperspectral image by an observation device connected to said client device;
the method comprises a step (a0) of learning, by data processing means of a server, parameters of said automatic classification model from a training database of hyperspectral images or of already classified spectra of colonies;
the microbial strain is a Staphylococcus aureus strain and the antimicrobial agent is methicillin.

A further aim of the invention is a system for determining the susceptibility of a microorganism to an antimicrobial agent, comprising at least one client device comprising data processing means, characterized in that said data processing means are configured to implement:
obtaining a hyperspectral image representing at least one colony of said microorganism in a sample,
determining a spectrum of the colony from the pixels of said hyperspectral image corresponding to said colony;
comparing said test spectrum with microbial classes, called “reference microbial class” hereafter, of a predetermined database, with said classes corresponding to a taxonomic level lower than the species and being learnt on at least one hyperspectral spectrum of a microbial strain, the database comprising, for each reference microbial class, the susceptibility to the antimicrobial agent of the reference microbial class;
determining the susceptibility of the microbial strain to the microbial agent as being that associated with the reference microbial class closest to the hyperspectral test spectrum.

According to one embodiment, the system further comprises an observation device for acquiring said hyperspectral image.

A further aim of the invention is a computer program product comprising code instructions for executing a method as described above for determining the susceptibility of a microorganism to an antimicrobial agent, when said program is executed on a computer.

A further aim of the invention is a computer device-readable storage means storing a computer program product comprising code instructions for executing a method as described above for determining the susceptibility of a microorganism to an antimicrobial agent.

### DESCRIPTION OF THE FIGURES

Further features and advantages of the present invention will become apparent upon reading the following description of a preferred embodiment. This description will be provided with reference to the appended drawings, in which:

FIG. 1 is a diagram of an architecture for implementing the method according to the invention;

FIG. 2a shows a first embodiment of a device for observing microorganisms in a sample used in an embodiment of the method according to the invention;

FIG. 2b shows a second embodiment of a device for observing microorganisms in a sample used in a preferred embodiment of the method according to the invention;

FIG. 3a shows an example of a colony spectrum of a class with resistance to an antimicrobial agent;

FIG. 3b shows an example of a colony spectrum of a class with sensitivity to an antimicrobial agent;

FIG. 4 shows the steps of a preferred embodiment of the method according to the invention;

FIG. 5 shows an example of a convolutional neural network architecture used in a preferred embodiment of the method according to the invention;

FIG. 6 shows a confusion matrix of a predictor based on a convolutional neural network according to the invention.

### DETAILED DESCRIPTION

### Architecture

The invention relates to a method for determining the susceptibility of a microorganism of a given species to an antimicrobial agent. Said microorganism is typically a bacterium, a mold or a yeast (the example of S. aureus will be used throughout the remainder of the description, but this could be E. coli, C. difficile, etc.), and said microbial agent is an antibiotic (in particular methicillin was then the antibiotic of choice for S. aureus, but also vancomycin, for example) or an antifungal agent relating to yeasts and molds.

As will be seen, this method can include a machine learning component, and notably a classification model selected from among a support vector machine (SVM) or a convolutional neural network (CNN).

More specifically, the method is a method for classifying an image, called hyperspectral image, of the microorganism, such that the input or learning data are of the image type, and represent at least one colony of said microorganism in a sample 22 (in other words, it involves images of the sample in which at least one colony, generally a plurality of colonies, is visible, i.e., detectable to the naked eye by a laboratory technician or detectable in the image by means of a segmentation algorithm that is per se known. By way of an example, a colony is detectable as soon as it reaches a size of more than 10 pixels in the image). The sample 22 is adapted to the culture of said microorganism, typically an agar poured in a Petri dish, even though it can be any culture medium or reactive medium. The notion of a hyperspectral image, denoted HSI image, will be referred to hereafter.

The present methods are implemented within an architecture as shown in FIG. 1, by virtue of a server 1 and a client device 2. The server 1 is the learning device (implementing the learning method) and the client device 2 is an operating device (implementing the method for determining the susceptibility of a microorganism to an antimicrobial agent), for example, a terminal of a doctor, a hospital or a microbiology laboratory.

It is quite possible for the two devices 1, 2 to be merged, but preferably the server 1 is a remote device, and the client device 2 is a consumer device, notably a desktop computer, a laptop, etc. The client device 2 is advantageously connected to an observation device 10, so as to be able to directly acquire said input image, typically for processing it directly; alternatively, the input image will be loaded on the client device 2.

In all cases, each device 1, 2 is typically a remote computing device linked to a local network or to a wide area network, such as the Internet, for exchanging data. Each device comprises data processing means 3, 4 of the processor type, and data storage means 5, 6, such as a computer memory, notably a permanent memory, for example, a flash memory or a hard disk, storing all the computer instructions for implementing the method according to the invention. The client device 2 typically comprises a user interface 7, such as a screen, for interaction.

The server 1 advantageously stores a database for the considered species, comprising a list of microbial strains belonging to the species, and, for each of said strains, comprising:
hyperspectral spectra for learning colonies of the strain, i.e., a set of already classified objects;
data relating to the sensitive or resistant nature of the strain to the microbial agent;
optionally data relating to the test conditions.

### Acquisition

Even if, as explained, the present method can directly take any hyperspectral image as input representing at least one colony of said microorganism in the sample 22, in particular a Petri dish in which an agar is poured forming a nutrient medium allowing the growth of microbial colonies after spreading a liquid sample containing one or more microbial strains, obtained in any manner, the present method preferably begins with a step (a) of obtaining the input image from data supplied by an observation device 10.

In a known manner, a person skilled in the art can use hyperspectral imaging techniques, in particular as described in international application WO 2019/122732.

A hyperspectral image is understood to mean an image comprising a large number of spectral channels, in particular at least seven, advantageously at least twenty, and potentially more than two hundred (the example of 223 channels will be used), compared with a conventional three-channel RGB image. In general, the device 10 is “simple” compared to that notably described by Park et al., in the document entitled, “Classification of Salmonella Serotypes with Hyperspectral Microscope Imagery”, in that it simply needs to be able to acquire an HSI image of the sample 22, and therefore does not require a microscope, the high magnification of which makes focusing difficult.

Two possible embodiments of the device 10, corresponding to FIGS. 2a and 2b, will now be described.

With reference to FIG. 2a, the device 10 is, for example, a reference hyperspectral imaging system, namely “Pika II” marketed by Resonon, Montana, USA. It advantageously comprises:
a “hyperspectral’ camera 18, made up of a digital sensor comprising an array of elementary sensors, for example, a CCD or CMOS type digital sensor, sensitive in a wavelength range, for example, [λmin; λmax]=[390 nm; 900 nm]; and of a light dispersive element or a spectrograph for selecting a wavelength to be acquired by the sensor;
an objective lens 20 for focusing, on the digital sensor of the camera 18, the optical image of the sample 22 for which a hyperspectral image is to be acquired;
front lighting 24, for example, made up of one or more allogeneic lamps, for example, 2 or 4 lamps, able to emit light in the range [λmin; λmax] and to provide uniform front lighting of the sample 22. For example, the lighting is of the white light lamp type;
rear lighting 26, for example, made up of a matrix of white light LEDs, in order to provide uniform rear lighting of the sample 22 in the range;
a carriage 28 supporting the sample 22 and allowing the sample to pass in front of the objective lens 20 in order to obtain a complete image by scanning.

The device 10 is configured, for example, to acquire the image of a region measuring 90 millimeters by 90 millimeters with a sampling rate of 160 micrometers (spatial resolution estimated at 300 micrometers) and with a spectral resolution of a few nanometers over the range [Amin, Amax]. 200 channels can be exceeded over a range of approximately 500 nm. In particular, the field of view and the depth of field of the objective lens 20 are selected so as to obtain images that can include complete colonies with a radius of up to 1 cm, preferably of up to 0.9 cm, and even more preferably of 0.5 cm.

The device 10 thus produces a digital HSI image of the light reflected by the sample 22, incorrectly called “hypercube” since it is actually three-dimensional: two spatial dimensions and one spectral dimension, with each pixel (or rather voxel due to the three-dimensional nature of the HSI image) representing the radiance measured at a point of the sample 22 for a spectral channel.

The radiance of a pixel, commonly called “luminous intensity”, in this case corresponds to the amount of incident light on the surface of the corresponding elementary sensitive site of the sensor of the camera 18 throughout the exposure duration, as is known per se in the field of digital photography, for example.

The device 10 can comprise on-board data processing means configured to process the HSI images produced by the camera 18 and/or to delegate everything to the client device 2.

These processing means in all cases are provided with the set of memories (RAM, ROM, cache, mass memory, etc.) for storing the images produced by the device 10, computer instructions for implementing the method according to the invention, parameters useful for this implementation and for storing the results of the intermediate and final computations. The client device 2 optionally comprises, as explained, a display screen 7 for displaying the final result of the method. Although a single processing unit is described, the invention obviously applies to processing carried out by several processing units (for example, an on-board unit in the camera 18 for pre-processing HSI images and the unit 4 of the client device 2 for implementing the remainder of the processing). Moreover, the interface 7 of the client device 2 can allow data to be entered that relates to the sample 22, notably the type of culture medium used when the prediction depends on the medium, for example, by means of a keyboard/mouse and a drop-down menu available to the operator, a barcode/QR code reader reading a barcode/QR code present on the Petri dish and comprising information relating to the sample 22, etc.

With reference to FIG. 2b, according to the second embodiment, the device 10 can alternatively comprise a camera 34, advantageously a high spatial resolution CMOS or CCD camera, coupled to a set of spectral filters 36, for example, disposed in front of the objective lens 20 between the objective lens 20 and the sensor of the camera 32. The set of filters 36 is made up of a number NF of distinct bandpass filters, each configured to only transmit light in part of the range [λmin; λmax], with a full width half maximum (FWHM) spectral width that is less than or equal to 50 nm, and preferably less than or equal to 20 nm. The set 36 is, for example, a filter wheel that can typically accommodate up to twenty-four different filters, which wheel is controlled by the data processing unit, which actuates it in order for said filters to pass in front of the camera, and to control image capturing for each of said filters.

### Method

The “classification” of an input HSI image involves determining at least one class from among a set of possible classes describing the images. The present method proposes using an automatic classification model for determining whether the microorganism to be tested belongs to one of the strains already listed in the database and not for directly determining the susceptibility of the microorganism to the antimicrobial agent or even whether the microorganism belongs to particular stereotypes, for example.

In particular, with reference to FIGS. 3a and 3b, which each represent a plurality of examples of spectra respectively for colonies of S. aureus resistant to methicillin (MRSA) and sensitive to methicillin (MSSA), it should be noted that the two spectra do not have exactly the same appearance, hence the possibility of a direct discriminating classification of the sensitive or resistant nature of a new colony of S. aureus. However, the inventors have found that the performance capabilities of such a susceptibility predictor are not sufficient in the field of clinical or industrial microbiology. For example, the performance capabilities of a Gaussian kernel SVM predictor plateau at 70% in BCR.

With reference to FIG. 4, the method comprises, after step (a) of obtaining the hyperspectral image, a step (b) of determining a spectrum of the colony from the pixels of said hyperspectral image corresponding to said colony.

The term “spectrum of the colony” is understood to mean a curve representing the light intensity measured to the scale of the colony as a function of the frequency. Mathematically, this is a size vector relating to the number of channels of the HSI image (i.e., 223 in the example provided herein).

Preferably, this spectrum is determined as the mean spectrum on the pixels of said hyperspectral image corresponding to said colony. Indeed, by way of a reminder, the HSI image includes a plurality of corresponding intensity values for each spatial pixel.

In this respect, step (b) advantageously comprises segmenting said hyperspectral image so as to detect said colony in the sample 22, then determining the spectrum, as is typically explained by averaging the intensity on a channel by channel basis over the segmented pixels. For example, step (b) comprises automatically detecting colonies (for example, by applying a filter selecting the round objects in the image, for example, a Hough transformation), and/or a manual step of selecting colonies by a laboratory technician, for example. In other words, there is a size vector of n=223 per pixel of the colony, and the mean of these vectors is made into a vector representing the colony. In practice, a colony generally extends over a zone of the HSI image with a maximum size of 11×11, so that the mean only needs to be provided for around a hundred vectors.

In the case whereby the hyperspectral image represents a plurality of colonies of said microorganism, the method according to the invention can be applied to each colony or to a set of colonies selected according to criteria relating to the size or the position in the culture medium, for example.

In general, the segmentation allows all the colonies of interest to be detected, by removing artefacts such as filaments or dust. The segmentation can be implemented in any known manner.

Step (b) advantageously comprises processing the spectrum, in particular smoothing and/or normalizing the spectrum:
smoothing involves removing the peaks that are probably artefacts, for example, by determining the moving mean;
normalizing aims to make the spectra comparable, notably using a standard normal variate (SNV) technique involving subtracting the mean from the spectrum and dividing it by its standard deviation.

It should be noted that if the learning database directly stores reference spectra, they preferably must have undergone the same smoothing and/or normalizing, if necessary.

### Classification

In a step (c), said spectrum of the colony (if necessary smoothed and/or normalized) is, as explained, directly classified by means of an automatic classification model from among a microbial class made up of the identity of the strains listed in the database. If a plurality of spectra has been determined, each spectrum can be classified, and the results can be aggregated.

The automatic classification model can be, as explained, a support vector machine (SVM) or a convolutional neural network (CNN). In the case of an SVM, an RBE (Radial Basis Function) kernel SVM is selected, for example.

In the case of a CNN, an architecture of the type shown in FIG. 5 is selected, for example, which is particularly suitable for implementing the present method. Conventionally, this architecture advantageously comprises a series of “convolutional blocks” made up of one or more 1D convolutional layers (since the input is not an image but the spectrum, i.e., a one-dimensional object), an activation layer (for example, the ReLU function) for increasing the depth of the feature maps, and a 1D pooling layer (in this case max pooling) allowing the size of the feature map to be reduced (generally by a factor of 2). It is noteworthy that two convolutional blocks can suffice, so that, highly preferably, the present CNN includes only two convolutional blocks.

Thus, in the example of FIG. 5, the CNN starts, as explained, with 12 layers distributed in 3 blocks. The first block takes the spectrum as input (thus forming a 223-size object), and includes a dual convolution+activation sequence increasing the depth to 16 and then a max pooling layer (it is also possible to use overall average pooling), with a 111×16-size feature map as output (the size is divided by two according to the spectral dimension).

The second block has an architecture identical to the first block and generates, as output from a new dual convolution+activation, a 111×32-size feature map (depth doubled) and, as output from the max pooling layer, a 55×32-size feature map (with a new reduction in the spectral size by a factor of two).

The third block has an architecture identical to the first two blocks and generates, as output from a new dual convolution+activation, a 55×32-size feature map (depth unchanged) and, as output from the max pooling layer, a 27×32-size feature map (with a new reduction in the spectral size by a factor of two).

At the output of the last convolutional block (in this case the third block), the CNN advantageously comprises a “flattening” layer that transforms the final feature map (containing the most “in-depth” information) output from this block into a vector (1-dimensional object). Thus, for example, the 27×32-size feature map switches to a vector that is 27*32=864. It will be understood that there is no limit to the sizes of maps/filters on any level, and that the aforementioned sizes are only examples.

Finally, conventionally, this results in one or more fully connected layers (FC, or “dense” layers, as indicated in FIG. 5) and optionally a final activation layer, for example, a softmax layer. In the example shown, a first dense layer transforms the 864-size vector into a smaller 256-size vector (which requires (864+1)*256=22, 1440 parameters, that is 90% of all the parameters of the CNN), and a second FC layer transforms the 864-size vector into a final vector of size C, with C being the desired total number of classes, that is 2, 5 or 11 in the aforementioned examples (which requires (256+1)*C parameters).

Preferably, the CNN is made up of (i.e., includes exactly) a sequence of convolutional blocks, then a flattening layer, and finally one or more fully connected layers.

Therefore, it can be seen that the total number of parameters is of the order of 200,000, which is remarkably low for a CNN (commonly there are several tens of millions of parameters). The present CNN therefore can be used by many client devices 2, including client devices with moderate computing resources.

Again, it should be noted that the term “direct classification” or “end-to-end” is understood to mean without pre-classification or separate extraction of at least one feature map of said colony: it is understood that the CNN naturally has internal states in the form of feature maps, but these maps are never returned to the outside of the CNN, with the CNN only having the result of the classification as output.

### Learning

Preferably, the method can comprise a step (a0) of learning, by the data processing means 3 of the server 1, the parameters of the automatic classification model from a learning database. Indeed, this step is typically implemented well upstream, in particular by the remote server 1. As explained, the learning database can include a certain amount of learning data, in particular hyperspectral images of colonies or even directly from the spectra, in all cases associated with their class (i.e., the identity of the microbial strains).

The learning for the model can be carried out in any way known to a person skilled in the art that is adapted to the selected model.

In all the embodiments, the parameters of the learnt model can be stored, if necessary, on data storage means 21 of the client device 2 for use in classification. It should be noted that the same model can be included on many client devices 2, yet only one learning step is necessary.

Preferably, the learning database for the considered microbial species is formed as follows. The following is carried out for each strain of said species:
producing several samples, preferably at least 3 samples, for example, several Petri dishes in which a nutrient agar has been poured that contains neither the antimicrobial agent nor a marking or staining agent, on which agar colonies of said strain have grown;
acquiring and storing hyperspectral spectra of at least one colony of each sample using a device as described in FIG. 2, preferably of several colonies per Petri dish and arranged at different distances from the center of the Petri dishes, and processing said spectra as described above. The spectra of the first two samples are used for learning the microbial classes of strains, with the spectra of the other samples (for example, the third sample when producing replicates) being used to test the performance capabilities of the learning. Optionally, the acquisition is also carried out on several samples using different capturing devices for capturing the variability of the spectra caused by differences in the features of the devices (for example, the variability of the light sources between devices, etc.);
phenotypic measuring of the susceptibility of the strain to the antimicrobial agent, for example, by means of a Vitek® 2 marketed by the Applicant or the use of an e-test or of a diffusion disc as is well known per se in the prior art, and the storage thereof;
genomic characterizing of the strain, for example, using complete sequencing of its genome and establishing a wgMLST profile as described in the document entitled, “MLST revisited: the gene-by-gene approach to bacterial genomics”, by Martin C. J. Maiden, Nature Reviews Microbiology, 2013, and storing this characterization.

### Updating the Database and the Predictor

When a strain is not listed in the database, as determined, for example, by the predictor according to the invention, which returns an uncertain classification in the pre-learnt microbial strain classes, characterizing said strain as described above is advantageously carried out. The genomic profile of the strain is advantageously compared with the stored genomic profiles in order to determine whether it is actually a strain different from those stored in the database. In this case, the data gathered for the strain are stored in the database and new learning is carried out as described above in order to incorporate a new microbial class corresponding to the unlisted strain.

### Computer Program Product

According to a second and a third aspect, the invention relates to a computer program product comprising code instructions for executing (in particular on the data processing means 3, 5 of the server 1 and/or of the client device 2) a method for determining the susceptibility of a microorganism to an antimicrobial agent, as well as computer device-readable storage means (a memory 4, 6 of the server 1 and/or of the client device 2) on which this computer program product is found.

### Preferred Applications of the Invention

The invention is advantageously incorporated in.
a method for epidemiologically monitoring strains of interest in a clinical or industrial environment (for example, a hospital department, a hospital as a whole, a group of hospitals, an agri-foodstuff plant, a potable water distribution facility, etc.) defining a given geographical zone, the database being made up of the strains sampled from said zone. The invention thus provides two important items of information for fighting nosocomial diseases or microbial contaminations not in accordance with food, environmental or manufacturing standards: the identification or non-identification of a newly sampled strain with a strain already seen in the geographical sampling zone and its susceptibility to the antimicrobial agent. These data associated with the sampling zones (for example, hospital room, production zone) allow, for example, investigations to be conducted concerning how these germs are disseminated within the hospital or within the production plant. It should be noted that within the context of hospital epidemiological monitoring, all the cultures can be used to feed the knowledge database, those implemented within the context of a microbiological diagnosis process, whether or not it integrates a susceptibility test, as well as those implemented in a hospital admission screening process on prevention range media. This invention thus provides the possibility of carrying out epidemiological monitoring of the various resistance clones present in the hospital;
a method for antibiotic therapy of a patient suspected of being infected with a pathogen. As is known per se, when a patient is suspected of being the victim of an infection, a combination of a broad spectrum of antibiotics is generally administered to them before knowing the identity and the antibiogram of the strain they are infected with, with the antibiotic therapy then being optionally modified once the antibiogram of the strain has been carried out. A microbial strain is usually characterized by proceeding through several successive steps of growing colonies on a Petri dish. By virtue of the invention, from the first sign of growth, a prediction of the identity of the strain and its susceptibility to one or more antimicrobials is available so that the clinician can adapt their therapy without waiting for the result of an antibiogram.

### EXAMPLE

The invention has been applied to predicting the susceptibility of 50 strains of Staphylococcus aureus to methicillin so as to define a predictor based on CNN identifying the MRSA and MSSA strains. The following table specifies, for each of the strains listed in the learning database, the number of colonies for which hyperspectral spectra were acquired and the susceptibility to methicillin.

| API number | Susceptibility | Total number |
|---|---|---|
| of the strain | to methicillin | of colonies |
| 1412368 | MSSA | 145 |
| 1412369 | MSSA | 108 |
| 412370 | MSSA | 134 |
| 1502039 | MSSA | 188 |
| 1412148 | MRSA | 179 |
| 1412174 | MRSA | 129 |
| 1412289 | MRSA | 86 |
| 1412330 | MRSA | 111 |
| 1412343 | MRSA | 118 |
| 1412350 | MRSA | 89 |
| 1502059 | MSSA | 139 |
| 1502063 | MSSA | 159 |
| 1502098 | MRSA | 187 |
| 1502125 | MSSA | 170 |
| 1412147 | MSSA | 128 |
| 1412162 | MSSA | 153 |
| 1412277 | MRSA | 116 |
| 1412300 | MRSA | 140 |
| 1412315 | MRSA | 166 |
| 1412316 | MRSA | 127 |
| 1502094 | MSSA | 209 |
| 1502121 | MSSA | 205 |
| 1503077 | MRSA | 142 |
| 1503078 | MRSA | 164 |
| 1412156 | MSSA | 213 |
| 1412157 | MSSA | 186 |
| 1412202 | MSSA | 78 |
| 1502123 | MSSA | 142 |
| 1412145 | MSSA | 161 |
| 1412153 | MSSA | 140 |
| 1412170 | MRSA | 200 |
| 1412171 | MSSA | 127 |
| 1412260 | MRSA | 108 |
| 1412262 | MRSA | 81 |
| 1412278 | MRSA | 122 |
| 1412303 | MRSA | 170 |
| 1412340 | MRSA | 123 |
| 1502073 | MSSA | 172 |
| 1412175 | MSSA | 128 |
| 1412177 | MSSA | 170 |
| 1502095 | MSSA | 206 |
| 1502124 | MSSA | 160 |
| 1412152 | MSSA | 125 |
| 1412197 | MSSA | 164 |
| 1412198 | MSSA | 148 |
| 1502041 | MSSA | 175 |
| 1412232 | MRSA | 154 |
| 1412233 | MRSA | 100 |
| 1412327 | MRSA | 91 |
| 1412353 | MRSA | 85 |

FIG. 6 illustrates the confusion matrix of a predictor of microbial strains of strains according to the neural network of FIG. 5. The global accuracy of the latter (global accuracy) is 88% and the mean accuracy per class (“balanced accuracy”) is 87%.

The invention claimed is:

1. A method for predicting the susceptibility of a microbial strain to an antimicrobial agent, the method comprising implementing, by a data processor of a client device, the steps of:
(a) obtaining a hyperspectral image between 390 nm and 900 nm representing at least one colony of the strain in a sample devoid of an antimicrobial agent;
(b) determining a spectrum, called “test spectrum” hereafter, of the colony from the pixels of the hyperspectral image corresponding to the colony;
(c) comparing the test spectrum with microbial classes, called “reference microbial class” hereafter, of a predetermined database, with the classes corresponding to a taxonomic level lower than the species and being learnt on at least one hyperspectral spectrum of a microbial strain, the database comprising, for each reference microbial class, the susceptibility to the antimicrobial agent of the reference microbial class; and
(d) determining the susceptibility of the microbial strain to the microbial agent as being that associated with the reference microbial class closest to the hyperspectral test spectrum.

2. The method as claimed in claim 1, wherein the steps of comparing and of determining are carried out by a predictor based on a monitored classification having the identity of the microbial strains of the database as reference microbial classes, with the phase of training the classification comprising:
(c1) acquiring hyperspectral spectra of various colonies for each microbial strain of the database; and
(c2) training the classification on the hyperspectral spectra of various colonies.

3. The method as claimed in claim 2, wherein the predictor is a convolutional artificial neural network.

4. The method as claimed in claim 1, wherein step (b) comprises segmenting the hyperspectral image so as to detect the colony in the sample.

5. The method as claimed in claim 1, wherein step (b) comprises smoothing the spectrum of the colony.

6. The method as claimed in claim 1, wherein step (a) comprises acquiring the hyperspectral image by an observation device connected to the client device.

7. The method as claimed in claim 1, comprising a step (a0) of learning, by the data processor of a server, parameters of the automatic classification model from a training database of hyperspectral images or of already classified spectra of colonies.

8. The method as claimed in claim 1, wherein the microbial strain is a Staphylococcus aureus strain and the antimicrobial agent is methicillin.

9. A system for determining the susceptibility of a microorganism to an antimicrobial agent, comprising at least one client device comprising a data processor, wherein the data processor is configured to implement the steps of:
(a) obtaining a hyperspectral image representing at least one colony of the microorganism in a sample;
(b) determining a spectrum of the colony from pixels of the hyperspectral image corresponding to the colony;
(c) comparing the test spectrum with microbial classes, called “reference microbial class” hereafter, of a predetermined database, with the classes corresponding to a taxonomic level lower than the species and being learnt on at least one hyperspectral spectrum of a microbial strain, the database comprising, for each reference microbial class, the susceptibility to the antimicrobial agent of the reference microbial class; and
(d) determining the susceptibility of the microbial strain to the microbial agent as being that associated with the reference microbial class closest to the hyperspectral test spectrum.

10. The system as claimed in claim 9, further comprising a hyperspectral imager acquiring the hyperspectral image.

11. A computer program product comprising code instructions determining the susceptibility of a microorganism to an antimicrobial agent, when the code instructions are executed on a computer, the code instructions operably:
(a) obtaining a hyperspectral image between 390 nm and 900 nm representing at least one colony of a strain in a sample devoid of the antimicrobial agent;
(b) determining a test spectrum of the colony from pixels of the hyperspectral image corresponding to the colony;
(c) comparing the test spectrum with reference microbial classes of a predetermined database, with the classes corresponding to a taxonomic level lower than the species and being learnt on at least the hyperspectral spectrum of the strain, the database comprising, for each of the reference microbial classes, the susceptibility to the antimicrobial agent of associated one of the reference microbial classes; and
(d) determining the susceptibility of the microbial strain to the microbial agent as being that associated with the one of the reference microbial classes closest to the hyperspectral test spectrum.

12. A computer device-readable storage means storing a computer program product comprising code instructions for executing a method as claimed in claim 1 for determining the susceptibility of a microorganism to an antimicrobial agent.

13. The method as claimed in claim 1, wherein step (b) comprises normalizing the spectrum of the colony.

14. The method as claimed in claim 1, wherein step (b) comprises smoothing and normalizing the spectrum of the colony.
